(19) United States
(12) Patent Application Publication
(10) Pub. No.: US 2012/0253092 A1
(43) Pub. Date: Oct. 4, 2012

(54) METHOD AND SYSTEM FOR REMOVAL OF FOULANT PRECURSORS FROM A RECYCLE STREAM OF AN OLEFINS CONVERSION PROCESS

(75) Inventors: CORNELIS F. VAN EGMOND, PASADENA, TX (US); DAVID LEUNG, SUGAR LAND, TX (US)

(73) Assignee: STONE & WEBSTER PROCESS TECHNOLOGY, INC., HOUSTON, TX (US)

(21) Appl. No.: 13/076,863

(22) Filed: Mar. 31, 2011

(51) Int. Cl. C07C 4/02; B01J 19/00; C10G 71/00

(52) U.S. Cl. 585/648; 208/106; 422/187

(57) ABSTRACT

A post fractionation process for removing heavy hydrocarbons from the C₄⁺ olefins conversion process reactor effluent, which act as foulants when recycled to the C₄⁺ olefins conversion reactor. This simple and effective process improves the run length of the reactor by reducing catalyst fouling, which also improves yields in a C₄⁺ olefins conversion process to light olefins. Essential to present invention is the efficient recycling of a hydrocarbon stream to the reactor, utilizing well proven equipment in a novel way to separate more valuable product from less desirable components in the recycle product stream.

### FIELD AND BACKGROUND OF THE INVENTION

The present invention relates to a post fractionation system and process for removing reactor effluent foulant in a hydrocarbon process stream. More particularly, the invention provides a system and process for improved olefins conversion by removing foulant from an olefins conversion rector feed stream.

Hydrocarbons are feedstocks for petrochemical industries. Olefins, diolefins, and paraffins are useful for preparing a wide variety of petrochemicals, especially light hydrocarbons, for example, ethylene, propylene, C₄ hydrocarbons (i.e., mixtures consisting of butanes, butylenes and butadienes) and heavier hydrocarbons, such as C₅ hydrocarbons and gasolines (generally, C₆+ hydrocarbons). These petrochemicals are typically produced by cracking petroleum feeds. A large number of methods described in the literature are directed to the production of olefins, such as steam pyrolytic cracking or catalytic cracking in processes such as fluid catalytic cracking (FCC) and deep catalytic cracking (DCC). With the high cost of suitable feedstocks for producing olefins, there is an increasing demand for energy efficient and lower capital production methods. Key to project economics is also reducing the loss of valuable reaction products during subsequent purification steps.

A variety of methods are available for producing light hydrocarbon streams, such as, steam cracking, fluid catalytic cracking, deep catalytic cracking, catalytic naphtha cracking and the conversion of methanol to olefins (MTO). These methods generate light hydrocarbons and heavy hydrocarbons.

Currently, C₄⁺ olefins conversion processes are finding greater application in areas of the world where end users are trying to produce more propylene from steam cracking and/or refining operations. Industry data indicate the demand for propylene production is strong and is expected to remain strong over the next 10 years in areas, such as, South America, China and South East Asia.

The prior art is replete with conventional C₄⁺ olefins conversion process methods to convert olefins to ethylene, propylene and the like. These methods include for example the use various types of zeolite catalyst to produce, inter alia, aromatic hydrocarbons and ethylene and propylene; methods of conversion of butene to ethylene and propylene; and methods of converting an olefin of 4-12 carbon atoms to ethylene and propylene. Included among these many methods for producing ethylene and propylene is a process for recycling a fraction comprising C₄ and higher hydrocarbons. However, in these processes, the C₄ and higher hydrocarbons are recycled to the reactor without removal of heavy hydrocarbons.

Previous attempts to improve ethylene and propylene yields by recycling hydrocarbon compounds of 4 or more carbon atoms, without removing heavy materials has been attempted. See, for example, U.S. Pat. Nos.: 5,026,935 and 5,043,522 to Leyshon et al.; and 7,754,934 to Takashi et al. However, adverse effects, such as, accelerated deposition of carbonaceous materials (coking) on the surface of the catalyst and equipment resulting in deterioration in catalytic activity and shorten the reactor cycle time decrease production efficiencies.

Prior art recycling of C₄⁺ , which includes C₅ and higher hydrocarbons, contains materials that act as “foulants” to the conversion process. Foulants, such as, C₆⁺ hydrocarbon materials have a predisposition for creating coke in the feed vaporization equipment feeding the reactor and the catalyst in the reactor. As such, these materials shorten the reactor cycle time, and catalyst life as well as reducing the benefits of the downstream processing units.

Accordingly, a need still exists for an efficient and effective system and process for improving the yields in a C₄⁺ olefins conversion process.

### SUMMARY OF THE INVENTION

The present invention is directed to a process for recovering olefins from a hydrocarbon feedstream, the process comprises: (i) reacting a hydrocarbon feedstream in a reactor to produce a reactor effluent containing hydrogen and C₁ to C₄⁺ hydrocarbons; (ii) separating the reactor effluent to obtain a C₃⁻ hydrocarbons overhead stream and a C₄⁺ hydrocarbons bottoms stream; (iii) separating the C₄⁺ hydrocarbons bottoms stream in a separator to obtain a C₄-C₅ hydrocarbon overhead recycle stream and C₆⁺ hydrocarbons bottoms stream for processing downstream; and (iv) recycling the C₄-C₅ overhead recycle stream to the reactor of step (i), wherein said C₄-C₅ overhead recycle stream is substantially free of C₆⁺ hydrocarbons.

Further, the present invention is directed to a system for recovering olefins from hydrocarbons comprising, sequentially connected, a hydrocarbon feed, a reactor for reacting said hydrocarbon feed to produce a reactor effluent containing C₁ to C₄⁺ hydrocarbons, at least one separator for separating C₃⁻ hydrocarbons from C₄⁺ hydrocarbons and at least one separator for simultaneously separating the C₄⁺ hydrocarbons into a C₄-C₅ hydrocarbons recycle stream and a C₆⁺ hydrocarbon stream for processing downstream, and recycling the C₄-C₅ hydrocarbons recycle stream to the reactor for cracking with the hydrocarbon feed, wherein said C₄-C₅ hydrocarbons recycle stream is substantially free of C₆⁺ hydrocarbons.

The process and system of the present invention improves the quality of the reactor effluent stream that would otherwise shorten the reactor cycle time and catalyst life as well as reducing the benefits of the downstream processing units. The inventive process and system not only increase product yields, but extend catalyst life, lengthen the time intervals between decoking of the reactor, and minimizing equipment sizes and capital investment.

### BRIEF DESCRIPTION OF THE SEVERAL VIEWS OF THE DRAWINGS

FIG. 1 A flow sheet showing an embodiment of construction of the system used for producing ethylene and propylene according to the process of the present invention.

FIG. 2 Represents a schematic flow diagram providing a non-limiting illustration of a process and a system in accordance with the present invention.

### DETAILED DESCRIPTION OF THE INVENTION

The particulars shown herein are by way of example and for purposes of illustrative discussion of the embodiments of the present invention only and are presented to provide what is believed to be the most useful and readily understood description of the principles and conceptual aspects of the present invention. In this regard, no attempt is made to show structural details of the present invention in more detail than is necessary for the fundamental understanding of the present invention, the description making apparent to those skilled in the art how the several forms of the present invention may be embodied in practice.

Essential to present invention is the efficient recycling of a hydrocarbon stream to the reactor, utilizing well proven equipment in a novel way to separate more valuable product from less desirable components in the recycle product stream. The invention substantially removes all of the heavier hydrocarbon compounds, i.e., C₆₊ materials , which have the greatest propensity for creating coke in the feed vaporization equipment feeding the reactor, as well as the catalyst of the reactor, from an olefins conversion reactor recycle stream.

Current cracking technologies for the production of light olefins (e.g. ethylene, propylene and, optionally, butylenes), gasoline and other cracked products such as light paraffins and naphtha can be classified into the two categories of thermal cracking (also known as steam cracking) and catalytic cracking. These technologies have been practiced for many years and are considered the workhorses for light-olefin production. It is well within the scope of the present invention to use the inventive recycling of a hydrocarbon stream to the reactor with both categories of cracking.

Cracked hydrocarbons typically comprise hydrogen, carbon monoxide, carbon dioxide, methane, acetylene, ethylene, ethane, methyl acetylene, propadiene, propylene, propane, butadienes, butanes, butenes, C₅ hydrocarbons, C₆-C₈ hydrocarbons, non-aromatics, benzene, toluene and other heavy hydrocarbons. These gases including olefins leave the reactor for further downstream processing. It is necessary to separate the useful olefins, e.g., ethylene and propylene, from the rest of the product gases.

When reference is made to a specific hydrocarbon stream, e.g., a C₄-C₅ stream, it is typically not intended that such reference is strictly limited to a pure C₄-C₅ stream, since other components, albeit in small amounts, may also be present. However, for purposes of the present invention, and according to embodiments of the present invention, the recycled materials (i.e., hydrocarbon recycle stream(s)), as disclosed herein, are substantially free of hydrocarbon compounds having more than 5 carbon atoms.

The C₄-C₅ overhead recycle stream although substantially free of C₆⁺ hydrocarbons materials, according to an embodiment of the invention the C₄-C₅ overhead recycle stream contains about 5 weight percent of the C₆₊ materials. According to another embodiment of the invention, the C₄-C₅ overhead recycle stream contains less than about 5 weight percent of the C₆₊ materials, and according to yet another embodiment of the invention, the C₄-C₅ overhead recycle stream contains less than about 2 weight percent of the C₆₊ materials.

In the process and system of the present invention, a hydrocarbon starting material containing more than 4 carbon atoms is used as a feedstock material for producing, inter alia, ethylene and propylene.

According to an embodiment of the invention, when the reaction mixture is separated into a fraction comprising hydrogen and hydrocarbons of 3 or less carbon atoms, i.e., C₃⁻, and hydrocarbons of 4 or more carbon atoms, i.e., C₄₊, the heavier byproducts comprising C₆₊ are first fractionated and purged from the C₄₊ recycle product stream, so as to create a substantially C₆₊ free C₄-C₅ recycle stream.

These heavier compounds, i.e., C₆₊, act as undesirable contaminants in the recycle product stream. In particular, the C₆ compounds and heavier olefinic and diolefinic compounds act as foulants and/or contaminants that cause equipment fouling, coking, interfere with polymerization reactions, and in some cases pose safety hazards. Moreover, the presence of C₆₊ materials in a stream for recycling to the reactor, create unfavorable conditions that reduce product yields by shortening reactor cycle time, reactor catalyst life, and also reduce the benefits of the downstream processing unit(s). Thus, the elimination and/or significant reduction of C₆₊ hydrocarbon materials in the recycle stream is greatly desired for increased efficiencies in the claimed process for recovering olefins from a hydrocarbon feedstream.

Although the inventors do not wish to be bound by any theory, of these contaminants, it is believed that fouling is the major cause of deterioration in catalyst performance. Fouling involves the deposition of carbonaceous matter on the catalyst surface (usually referred to as coke). This has at least two deleterious results: (a) physical blockage of the catalytically active surface sites and, on a larger scale, (b) plugging of catalyst pores such that diffusion of the feedstock through the pores to the active sites is impeded.

Coke fouling of catalytic sites usually occurs via adsorption of certain molecular species (referred to as coking precursors), which are bound strongly to the sites and may be easily polymerized and/or condensed to form large molecular structures.

The present inventors have found that by removing these higher molecular weight species, while still recycling the C4 and C5 components, a desirable process in which high yields of propylene (and ethylene) can be obtained with significant reduction in fouling is simply and readily obtained. The higher molecular weight compounds can then be processed downstream.

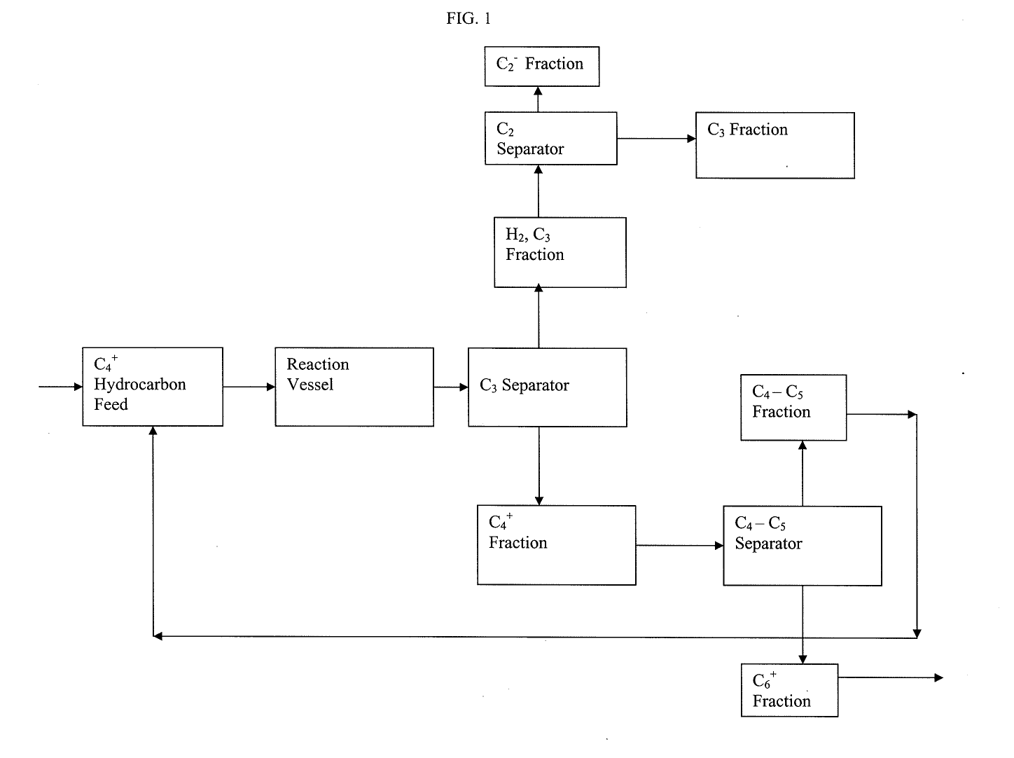

FIG. 1 presents one particular embodiment of the recycle reaction system for removal of foulant precursors from the recycle product stream of an olefins conversion process when C₄₊ hydrocarbon feed starting material is reacted in a reaction vessel, e.g., thermal cracking or catalytic cracking, utilizing conventional equipment, temperatures and pressures known in the art. A reaction mixture effluent (a mixture of hydrogen and hydrocarbons of 1 or more carbon atoms) is separated into an overhead fraction containing mainly hydrogen and hydrocarbons of 1-3 carbon atoms (hereinafter referred to as “H₂—C₃⁻ fraction”) and a bottoms fraction containing hydrocarbons of 4 or more carbon atoms (hereinafter referred to as “C₄⁺ fraction”). As the apparatus used for the separation of the H₂—C₃⁻ fraction and C₄⁺ , there may be used, for example, a depropanizer, a distillation column, a flash drum (vapor-liquid separator), etc., wherein the depropanizer is preferred. Ethylene and propylene are recovered from the resulting H₂—C₃⁻ fraction. On the other hand, the C₄⁺ bottoms fraction is separated into an overhead C₄-C₅ recycle stream and recycled back to the C₄⁺ hydrocarbon feed starting material and utilized as a part of the starting material, which is sent to the reaction vessel. The C₄-C₅ recycle stream is substantially free of C₆⁺ hydrocarbons. The bottom C₆⁺ hydrocarbons are transported downstream for further processing, as will be apparent to those of ordinary skill in the art. As the apparatus used for separation C₄-C₅ hydrocarbons from the C₆⁺ , there may be used, for example, a depentanizer, a distillation column, a flash drum (vapor-liquid separator), etc., wherein the depentanizer is preferred.

Further, the H₂—C₃⁻ fraction may be separated into a fraction containing mainly hydrogen and a hydrocarbon of 1-2 carbon atoms (hereinafter referred to as “C₂⁻ fraction”) and a fraction containing mainly a hydrocarbon of 3 carbon atoms (hereinafter referred to as “C₃ fraction”). As the apparatus used for separation (C₂ separator), there may be used, for example, a deethanizer, a distillation column, a flash drum (vapor-liquid separator), etc., wherein the deethanizer is preferred. When propylene is selectively produced, a part of the C₂⁻ fraction can be recycled to the reaction vessel and ethylene in the C₂⁻ fraction can be utilized as a part of the starting feedstock material.

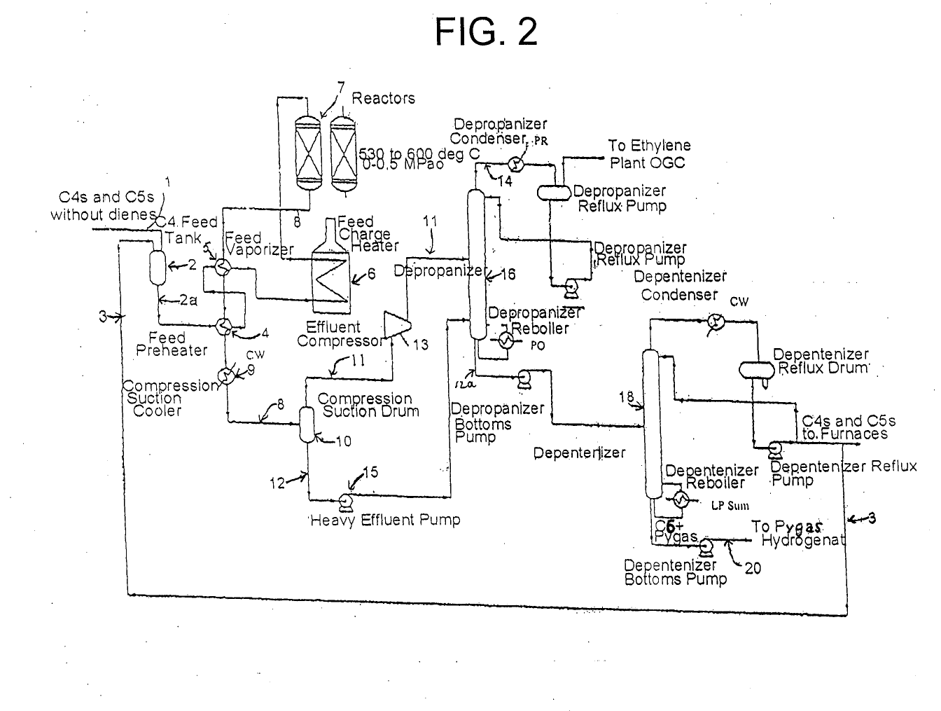

FIG. 2 presents a particular embodiment of the recycle reaction system for removal of foulant precursors from the recycle stream of an olefins conversion process when C₄₊ hydrocarbon feed starting material is reacted in a reaction vessel, e.g., thermal cracking or catalytic cracking, as known in the art.

Hydrocarbon feeds entering the system by way of inlet 1 and will include hydrocarbon feeds, like raw C₄₊ hydrocarbons and heavy hydrocarbon feeds including pygal/gasoline (containing C₆+hydrocarbons) feeds, light reformates, and optionally other C₄⁺ hydrocarbon feeds. Inlet 1 directs the hydrocarbon feed to C₄⁺ feed tank 2 to combine with a C₄-C₅ recycle stream 3 in C₄⁺ feed tank 2 to provide a combined hydrocarbon recycle feed 2a. The combined hydrocarbon recycle feed is sent to a feed preheater 4. After leaving feed preheater 4 the combined hydrocarbon recycle feed 2a is directed to a feed vaporizer 5 prior to entering a feed charge heater 6. The combined hydrocarbon recycle product feed 2a is heated in feed charge heater 6 to a temperature that ranges from about 530° C. to about 600° C., preferably from about 530° C. to about 550° C. and a pressure that ranges from about 1 bar a to about 5 bar a, preferably from about 1 bar a to about 2 bar a. The combined hydrocarbon recycle product feed 2a is then forwarded to the reactor 7 wherein the combined hydrocarbon recycle feed is appropriately reacted at conditions 530° C. and 1 bar a range. The combined hydrocarbon recycle feed 2a is reacted to provide a reactor effluent 8 that is forwarded to a compression suction cooler 9 and then sent to compression suction drum 10 for separating the reactor effluent 8 in to an overhead reactor effluent stream 11 and a bottom reactor effluent stream 12.

Overhead reactor effluent stream 11 passes through effluent compressor 13 prior to entering depropanizer 16 and bottom reactor effluent stream 12 passes through heavy effluent pump 15 prior to entering depropanizer 16. Overhead reactor effluent stream 11 combines with bottom reactor effluent stream 12 in depropanizer 16 to provide a C₃⁻ overhead stream 14 for further processing downstream and a C₄⁺ bottoms stream 12a that is forward to depentanizer 18 for separation. Depending on the desired product slate, a portion of the C3− overhead stream 14 can be recycled back to the hydrocarbon feeds entering the system. The C₄⁺ bottoms stream 12a of depropanizer 16 sent to depentanizer 18 to provide the C₄-C₅ recycle stream 3 and a C6+products bottoms stream 20. The C₆⁺ products bottom stream 20 is further processed downstream, however, the substantially C₆⁺free C₄-C₅ recycle stream 3 is recycled back to the C₄⁺ feed tank 2 and combined with hydrocarbon feed.

According to the invention, the C₄-C₅ recycle product stream 3, is substantially free from C₆⁺ hydrocarbons when recycled back to the hydrocarbon feeds entering the system.

The present invention can be illustrated by the following prophetic example. This example is intended for illustrative purposes only and should not be considered as limiting the invention. FIG. 2 illustrates an embodiment of the disclosed system wherein C₄⁺ hydrocarbons via inlet 1 are combined in C₄⁺ feed tank 2 and combined with C₄-C₅ recycle stream 3 and provide a combined hydrocarbon recycle feed 2a. The combined hydrocarbon recycle product feed 2a is reacted in a fixed bed vapor phase C₄-C₅ olefins conversion reactor 7. After reacting the combined hydrocarbon recycle product feed 2a to provide reactor effluent 8, the reactor effluent 8 is sent to compression suction drum 10 for separating the reactor effluent 8 into an overhead reactor effluent stream 11 and a bottom reactor effluent stream 12. The example contemplates the post reactor separation towers utilizing cooling water condensers. This preferred operation will set the recycle stream pressure. Overhead reactor effluent stream 11 combines with bottom reactor effluent stream 12 in depropanizer 16 to provide a C₃⁻ overhead stream 14 for further processing downstream and a C₄+ bottoms stream 12a that is forward to depentanizer 18 for separation to provide the C₄-C₅ recycle stream 3 and a C₆⁺ products bottoms stream 20. The pressure in the recycle stream 3 can be as high as 18 bar a and as low as 1 bar a, however, the recycle stream 3 pressure is preferably set at about 10 bar a. The C₄-C₅ recycle stream 3 is substantially C₆⁺ free and is recycled back to the C₄⁺ feed tank 2 and combined with hydrocarbon feed for reacting in reactor 7.

It is further understood that although the invention has been specifically described with reference to particular means and embodiments, the foregoing description is that of preferred embodiments of the invention. The invention, however, is not limited to the particulars disclosed but extends to all equivalents, and various changes and modifications may be made in the invention without departing from the spirit and scope thereof.

What is claimed is:

1. A process for recovering olefins from a hydrocarbon feedstream comprising:
(i) reacting a hydrocarbon feedstream in a reactor to produce a reactor effluent containing hydrogen and C₁ to C₄⁺ hydrocarbons;
(ii) separating the reactor effluent to obtain a C₃⁻ hydrocarbons overhead stream and a C₄⁺ hydrocarbons bottoms stream;
(iii) separating the C₄⁺ hydrocarbons bottoms stream to obtain a C₄-C₅ hydrocarbon overhead recycle stream and a C₆⁺ hydrocarbons bottoms stream for processing down stream; and
(iv) recycling the C₄-C₅ overhead recycle stream to the reactor of step (i), wherein said C₄-C₅ overhead recycle stream is substantially free of C₆⁺ hydrocarbons.

2. The process of claim 1, wherein the hydrocarbon feedstream comprises C₄-C₈ hydrocarbons containing olefins.

3. The process of claim 1, wherein the hydrocarbon feedstream comprises naphtha.

4. The process of claim 1, wherein the reactor effluent is compressed before separation into the substantially C₃⁻ hydrocarbons overhead stream for processing down stream and the substantially C₄⁺ hydrocarbons bottoms stream.

5. The process of claim 1, wherein said reactor effluent is separated in a depropanizer to obtain a C₃⁻ hydrocarbons overhead stream and a C₄⁺ hydrocarbons bottoms stream.

6. The process of claim 1, wherein said C₄⁺ hydrocarbons bottoms stream is separated in a depentanizer to obtain a C₄-C₅ hydrocarbon overhead recycle product stream and a C₆⁺ hydrocarbons bottoms stream.

7. The process of claim 1, wherein the C₄-C₅ hydrocarbon overhead recycle stream is recycled to the hydrocarbon feedstream of step (i) prior to reacting in the reactor.

8. The process of claim 1, wherein said C₄-C₅ recycle stream has pressure from about 1 bar a to about 18 bar a.

9. The process of claim 1, wherein said C₄-C₅ recycle stream has pressure of about 10 bar a.

10. The process of claim 1, wherein said C₄-C₅ recycle stream contains less than about 2 weight percent of the C₆⁺ materials.

11. A system for recovering olefins from hydrocarbons comprising, sequentially connected, a hydrocarbon feed, a reactor for reacting said hydrocarbon feed to produce a reactor effluent containing hydrogen and C₁ to C₄⁺ hydrocarbons, at least one separator for separating C₃⁻ hydrocarbons from C₄⁺ hydrocarbons and at least one separator for simultaneously separating the C₄⁺ hydrocarbons into a C₄-C₅ hydrocarbons recycle stream and a C₆⁺ hydrocarbon stream for processing downstream, and recycling the C₄-C₅ hydrocarbons recycle stream to the reactor for cracking with the hydrocarbon feed, wherein said C₄-C₅ hydrocarbons recycle stream is substantially free of C₆⁺ hydrocarbons.

12. The system of claim 11, wherein the hydrocarbon feedstream comprises C₄-C₈ hydrocarbons containing olefins.

13. The system of claim 11, wherein the hydrocarbon feedstream comprises naphtha.

14. The system of claim 11, wherein the reactor effluent is compressed before separation into the substantially C₃⁻ hydrocarbons overhead stream for processing down stream and the substantially C₄⁺ hydrocarbons bottoms stream.

15. The system of claim 11, wherein said reactor effluent is separated in a depropanizer to obtain a C₃⁻ hydrocarbons overhead stream and a C₄⁺ hydrocarbons bottoms stream.

16. The system of claim 11, wherein said C₄⁺ hydrocarbons bottoms stream is separated in a depentanizer to obtain a C₄-C₅ hydrocarbon overhead recycle product stream and a C₆⁺ hydrocarbons bottoms stream.

17. The system of claim 11, wherein the C₄-C₅ hydrocarbon overhead recycle stream is recycled to the hydrocarbon feedstream of step (i) prior to reacting in the reactor.

18. The system of claim 11, wherein said C₄-C₅ recycle stream has pressure from about 1 bar a to about 18 bar a.

19. The system of claim 11, wherein said C₄-C₅ recycle stream has pressure of about 10 bar a.

20. The system of claim 11, wherein said C₄-C₅ recycle stream contains less than about 2 weight percent of the C₆⁺ materials.
